SEQUENCE LISTING (8 sequences)

SEQ ID NO: 1 - protein, 21 aa, synthetic construct
  REGION 1..21: note = Laboratory synthesized peptide
AGYLLGKINLKALAALAKKIL

SEQ ID NO: 2 - protein, 15 aa, synthetic construct
  REGION 1..15: note = Laboratory synthesized peptide
RRWRRWNRFNRRRCR

SEQ ID NO: 3 - protein, 12 aa, synthetic construct
  REGION 1..12: note = Laboratory synthesized peptide
GRKKRRQRRRPQ

SEQ ID NO: 4 - protein, 16 aa, synthetic construct
  REGION 1..16: note = Laboratory synthesized peptide
RQIKIWFQNRRMKWKK

SEQ ID NO: 5 - protein, 16 aa, synthetic construct
  REGION 1..16: note = Laboratory synthesized peptide
  MOD_RES 1: note = ACETYLATION
  MOD_RES 16: note = AMIDATION
RQIKIWFQNRRMKWKK

SEQ ID NO: 6 - protein, 18 aa, synthetic construct
  REGION 1..18: note = Laboratory synthesized peptide
  MOD_RES 1: note = Modified Residue 1 Cysteine or cystine
CRQIKIWFQNRRMKWKKC

SEQ ID NO: 7 - protein, 12 aa, synthetic construct
  REGION 1..12: note = Laboratory synthesized peptide
  MOD_RES 1: note = Modified Residue 1 Cysteine or cystine
CKIWFQNRRMKC

SEQ ID NO: 8 - protein, 12 aa, synthetic construct
  REGION 1..12: note = Laboratory synthesized peptide
  MOD_RES 1: note = Modified Residue 1 Cysteine or cystine
CKIWFANRRMKC

(19) United States
(12) Patent Application Publication
(10) Pub. No.: US 2025/0289849 A1
(43) Pub. Date: Sep. 18, 2025

(54) PEPTIDE TO TREAT ALPHA-SYNUCLEIN AMYLOID BASED DISORDERS

(71) Applicant: COUNCIL OF SCIENTIFIC & INDUSTRIAL RESEARCH, New Delhi (IN)

(72) Inventors: Deepak Kumar SHARMA, Chandigarh (IN); Arpit GUPTA, Chandigarh (IN); Gajendra Pal Singh RAGHAVA, New Delhi (IN); Ankur GAUTAM, Chandigarh (IN); Anil KOUL, Chandigarh (IN); Priyanka SINGH, Chandigarh (IN)

(73) Assignee: COUNCIL OF SCIENTIFIC & INDUSTRIAL RESEARCH, New Delhi (IN)

(21) Appl. No.: 18/848,315

(22) PCT Filed: Mar. 21, 2023
(86) PCT No.: PCT/IN2023/050274

§ 371 Date: Sep. 18, 2024

(30) Foreign Application Priority Data
Mar. 23, 2022 (IN) 202211016557

(51) Int. Cl. C07K 7/08; A61K 38/00; A61P 25/28; G01N 33/68

(52) U.S. Cl. CPC C07K 7/08; A61P 25/28; G01N 33/6896; A61K 38/00; G01N 2333/4709; G01N 2800/52

(57) ABSTRACT

The present invention relates to a cell-penetrating peptide and its derivatives to inhibit α-synuclein fibrillation. The present invention specifically relates to a peptide-based inhibitor of Parkinson's Disease. The present invention discloses the identification of the peptides permeable to blood brain barrier for inhibition. α-synuclein fibril formation is observed in the presence of peptides as indicated by SEQ. ID-7, SEQ. ID-8 wherein the SEQ. ID-7, SEQ. ID-8 are truncated versions of SEQ. ID-4 having the homology of 83% and 75% respectively. It provides a peptide having an amino acid sequence of Formula 1. The present invention also provides a pharmaceutical composition comprising a peptide of Formula 1 along with the pharmaceutically acceptable excipient(s) having inhibitory activity against β-sheet polymerisation of amyloidogenic proteins. The analysis showed that peptides corresponding to SEQ. ID-7, SEQ. ID-8 are better inhibitors than SEQ. ID-4 against α-synuclein fibrillation.

### TECHNICAL FIELD OF THE INVENTION

The present invention relates to a peptide for treating α-synuclein amyloid based disorders such as Parkinson's Disease (PD) and other α-synucleinopathies.

This invention relates to the use of small peptides that are cell penetrating in nature, in inhibiting the process of conversion of intrinsically disordered proteins such as α-synuclein into β-sheet rich amyloid fibrils, and thus could be used as potent lead molecule for the treatment of diseases known as α-synucleinopathies.

### BACKGROUND OF THE INVENTION

The current study focuses on the development of inhibitors of the process of α-synuclein fibrillation in which cytotoxic fibrils are formed by intrinsically disordered monomeric protein α-synuclein. The α-synuclein is known to form amyloid fibrils and the deposition of such protein inclusions in cells mediate cytotoxicity which is associated with not only Parkinson's Disease (PD), but also many discases collectively known as α-synucleinopathics. α-synuclein as a main component of the Lewy body, a defining pathological characteristic of PD, has been reported by Spillantini, M. G. et al. [alpha]-Synuclein in Lewy bodies. Nature 388, 839-840 (1997). Spillantini, M. G., Crowther, R. A., Jakes, R., Hasegawa, M. &Goedert, M. α-Synuclein in filamentous inclusions of Lewy bodies from Parkinson's disease and dementia with Lewy bodies. Proceedings of the National Academy of Sciences of the United States of America 95, 6469-6473 (1998) doi: 10.1073/PNAS.95.11.6469, has reported that α-synuclein forms the major filamentous component of Lewy bodies and Lewy neurites. According to Centre for Disease Control (CDC) report, PD is the 14^{th} leading cause of death in U.S. (National Vital Statistics Report, 2014). PD is caused due to death of dopaminergic neurons, which are responsible for controlling muscle activity. The key pathological feature of PD is the formation of abnormal aggregate of proteins known as lewy bodies.

The α-synuclein is the main component of lewy bodies deposited in dopaminergic neurons of PD patients. α-Synuclein is a member of the Synuclein family of proteins abundantly present in pre-synaptic terminals of neuronal tissues. The Synuclein members are characterized by the presence of 5 or 6 imperfect repeats of motif KTKEGV at the N-terminus, a central hydrophobic non-amyoid beta component (NAC) domain and an acidic carboxy terminal tail. It is believed that the protofibrils that are formed during conformational transition of native α-synuclein into fibrils cause toxicity (Bucciantini, M. et al. Inherent toxicity of aggregates implies a common mechanism for protein misfolding diseases. Nature 416, 507-511, 2002), and thus strategies to block the process of fibrillation would lead to effective therapeutics against amyloid based disorders. One of the main challenges in the design of such inhibitors is that many of the amyloid based disorder are neurodegenerative diseases and thus potential inhibitors must be able to cross blood brain barrier. The design of such molecules that have dual properties of inhibition of fibrillation and ability to cross blood-brain-barrier remains a challenge.

Over the last decade, cell-penetrating peptides (CPPs) have been emerged as a versatile drug delivery system (Heitz, F., Morris, M. C. & Divita, G. Twenty years of cell-penetrating peptides: from molecular mechanisms to therapeutics. British Journal of Pharmacology 157, 195-206, 2009, doi: 10.1111/j.1476-5381.2009.00057.x). Lindgren, M. &Langel, Ü. in Cell-Penetrating Peptides: Methods and Protocols (edÜloLangel) 3-19 (Humana Press, 2011) reported CPPs constitute a family of small peptides, (<30 amino acids), often have a net positive charge and have inherent ability to traverse the plasma membrane without causing significant membrane damage. Owing to their cell-penetrating ability, CPPs have been used widely for the intracellular delivery of variety of cargoes including proteins (Nasrollahi, S. A., Fouladdel, S., Taghibiglou, C., Azizi, E. & Farboud, E. S. A peptide carrier for the delivery of elastin into fibroblast cells. International Journal of Dermatology 51, 923-929, (2012), doi: 10.1111/j.1365-4632.2011.05214.x.), peptides (Boisguerin, P., Giorgi, J.-M. &Barrere-Lemaire, S. CPP-conjugated Anti-apoptotic Peptides as Therapeutic Tools of Ischemiareperfusion Injuries. Current Pharmaceutical Design 19, 2970-2978, 2013; Soto-Jara, C. U.S. Pat. No. 5,948,673, 1999; Frangione, B. U.S. Pat. No. 7,473,482, 2009; El-Agnaf. O. M. A. U.S. Patent 2,004,009,625, 2004), small molecules (Shi, N.-Q., Gao, W., Xiang, B. & Qi. X.-R. Enhancing cellular uptake of activable cell-penetrating peptide-doxorubicin conjugate by enzymatic cleavage. International Journal of Nanomedicine 7, 1613-1621, 2012, doi: 10.2147/ijn.s30104; Nordstedt, C. U.S. Pat. No. 6,331,440, 2001), nucleic acid (Lehto, T., Kurrikoff, K. Langel, Ü. Cell-penetrating peptides for the delivery of nucleic acids. Expert Opinion on Drug Delivery 9, 823-836,201. doi: 10.1517/17425247.2012.689285 and Margus, H., Padari, K. &Pooga. M. Cell-penetrating Peptides as Versatile Vehicles for Oligonucleotide Delivery. Molecular Therapy 20, 525-533, 2012, doi: 10.1038/mt.2011.284), nanoparticles etc. (Dekiwadia, C. D., Lawrie, A. C. & Fecondo, J. V. Peptide-mediated cell penetration and targeted delivery of gold nanoparticles into lysosomes. Journal of Peptide Science 18, 527-534, 2012, doi: 10.1002/psc.2430) both in vitro and in vivo.

Though CPPs are being used extensively for the delivery of different therapeutic agents in order to treat variety of diseases including cancer, psoriasis, microbial infections (Fonseca, S. B., Pereira, M. P. & Kelley. S. O. Recent advances in the use of cell-penetrating peptides for medical and biological applications. Advanced Drug Delivery Reviews 61, 953-964, 2009 doi: http://dx.doi.org/10.1016/j.addr.2009.06.001), their applications in the treatment of neurodegenerative diseases is relatively underexplored. Interestingly, a few studies such as Zou, L.-L., Ma, J.-L., Wang, T., Yang, T.-B. & Liu, C.-B. Cell-Penetrating Peptide-Mediated Therapeutic Molecule Delivery into the Central Nervous System. Current Neuropharmacology 11, 197-208, 2013 doi: 10.2174/1570159X11311020006, Xia, H. et al. Penetratin-functionalized PEG-PLA nanoparticles for brain drug delivery. International Journal of Pharmaceutics 436, 840-850, 2012, doi: http://dx.doi.org/10.1016/j.ijpharm.2012.07.029, Spencer, B. et al. α-synuclein conformational antibodies fused to penetratin are effective in models of Lewy body disease. Annals of Clinical and Translational Neurology 3, 588-606, 2016, doi: 10.1002/acn3.321 and Lin, T. et al. Nose-to-brain delivery of macromolecules mediated by cell-penetrating peptides. Acta Pharmaccutica Sinica. B 6, 352-358, 2016 doi: 10.1016/j.apsb.2016.04.001 have reported the ability of CPPs to cross the BBB, and thus making them potential candidate for delivery of therapeutic agents for access to diseased region of the brain. Penetratin is a well-known CPP, which was discovered more than two decades back in 1994 (Derossi, D., Joliot, A. H., Chassaing, G. &Prochiantz, A. The third helix of the Antennapedia homcodomain translocates through biological membranes. The Journal of biological chemistry 269, 10444-10450, 1994). It is 16 amino acid long polypeptide and derived from third α-helix of the homcodomain of Antennapedia (segments 43-58), a Drosophila homcoprotein (Derossi, D. et al. Cell internalization of the third helix of the Antennapedia homcodomain is receptor-independent. The Journal of biological chemistry 271, 18188-18193, 1996). Penetratin is rich in positively charged residues and has been increasingly used as a potential vehicle for various drug as well as biomolecule delivery applications (Fonseca, S. B., Pereira, M. P. & Kelley, S. O. Recent advances in the use of cell-penetrating peptides for medical and biological applications. Advanced drug delivery reviews 61, 953-964, 2009, doi: 10.1016/j.addr.2009.06.001 and Stewart, K. M., Horton, K. L. & Kelley, S. O. Cell-penetrating peptides as delivery vehicles for biology and medicine. Organic & biomolecular chemistry 6, 2242-2255, 2008, doi: 10.1039/b719950c). Meade, A., Meloni, B., Mastaglia, F. &Knuckey, N. The application of cell penetrating peptides for the delivery of neuroprotective peptides/proteins in experimental cerebral ischacmia studies. Journal Of Experimental Stroke and Translational Medicine 2, 22-40, 2009 has reported Penetratin to cross blood brain barrier as well.

### OBJECTIVES OF THE INVENTION

The main objective of the invention is to provide a peptide to treat α-synuclein amyloid based disorders. The invention provides inhibitors of the process of fibrillation formed by intrinsically disordered protein α-synuclein and hence capable of curing disorders and diseases relating to α-synuclein amyloid such as pd and other α-synucleinopathies.

It is a further object of the present invention to identify and provide peptide based inhibitors of α-synuclein fibrillation with potential to cross Blood-brain-barrier.

### SUMMARY OF THE INVENTION

Accordingly, the present invention provides a peptide to treat α-synuclein amyloid based disorders more particularly it pertains to cell-penetrating peptide of Formula 1 which inhibits fibrillation of an intrinsically disordered protein, and thus could be used as potent lead molecule for the treatment of α-synuclein amyloid based disorders such as PD and other α-synucleinopathies.

Thus, in one embodiment, this invention provides a peptide having an amino acid sequence of Formula 1 X₁[RQI]ₘKIWFX₂NRRMK[WKK]ₘX₃ as well as salt thereof for inhibition of α-synuclein protein aggregation involved in amyloid associated disorders, wherein X₁ and X₃ can either be absent or is a Cysteine, X₂ can either be Gln or Ala and m can have value 0 or 1.

In particularly preferred embodiments the peptide according to formula 1, wherein X₁ and X₃ can either be Cysteine or Cystine.

In particularly preferred embodiments the peptide according to formula 1, wherein where m=0 or 1.

In particularly preferred embodiments the peptide according to formula 1, wherein m=0 or 1, X₂ can be Gln or Ala.

In another embodiment the peptide according to formula 1 further comprising groups selected from an acetyl group at N-terminus and an amide group at C-terminus.

In another embodiment the peptide according to Formula 1 for use as a medicament.

In another embodiment pharmaceutical composition comprising a peptide of formula 1 optionally along with pharmaceutically acceptable excipient(s).

In another embodiment the pharmaceutical composition comprising a peptide of formula 1 optionally along with pharmaceutically acceptable excipient(s) for treating PD.

In another embodiment the pharmaceutical composition comprising a peptide of formula 1 having inhibitory activity against β-sheet polymerisation of amyloidogenic proteins.

In another embodiment the pharmaceutical composition comprising a peptide of formula 1 having inhibitory activity against β-sheet polymerisation of amyloidogenic proteins wherein the protein is α-synuclein.

Another embodiment provides a method of inhibition of α-synuclein amyloid fibrillation by amino acid sequence of Formula 1 and salt thereof.

Another embodiment relates to use of peptide having an amino acid sequence of Formula 1 and salt thereof for inhibition of α-synuclein protein aggregation.

Another embodiment relates to use of peptide having an amino acid sequence of Formula 1 and salt thereof for the preparation of a medicament for treatment of disorders and diseases relating to α-synuclein amyloid such as Parkinson's Disease and other α-synucleinopathies.

### BRIEF DESCRIPTION OF THE DRAWINGS

FIG. 1. SEQ.ID.-4 inhibits α-synuclein fibrillation. The α-synuclein fibrillation was monitored using ThioflavinT (ThT), a dye that specifically binds to amyloids. The binding of ThT results in increase of its fluorescence intensity at 482 nm. Purified α-synuclein (400 μM) was incubated at 37° C. with shaking in the presence and absence of equimolar concentration of indicated peptides. Amyloid formation was determined by monitoring ThT fluorescence at different time intervals.

FIG. 2. SEQ.ID.-4 promotes α-synuclein solubilization (A) The α-synuclein fibrillation assay was performed in the presence and absence of SEQ.ID.-4, as described above. The aliquots were collected before (0 hrs) or after (7 hrs) fibril formation, and fractioned into supernatant and pellet by centrifugation at 4000 rpm for 1 min. The fractions were incubated with SDS loading buffer at 99° C. for 15 min, and further separated by 15% SDS-PAGE (Sodium dodecyl-sulfate polyacrylamide gel electrophoresis). Shown are supernatant and pellet fraction from α-synuclein without (lane 1, 2) and with SEQ.ID.-4 (lane 3, 4) at 0 hrs, and α-synuclein without (lane 5, 6) and with SEQ.ID.-4 (lane 7, 8) after 7 hrs of incubation at 37° C. (B) The secondary structural content of α-synuclein (12.5 μM) in the presence and absence of equimolar concentration of SEQ.ID.-4 was analysed by CD. In each case average of three spectra is reported. As expected, CD spectrum corresponding to α-synuclein alone shows characteristic of intrinsically disordered protein. The α-synuclein aliquots collected after 7 hrs of incubation at 37° C. show the presence of β-sheet content. As seen similar aliquots from reaction containing α-synuclein in the presence of SEQ.ID.-4 show CD spectra similar to that observed for α-synuclein alone.

FIG. 3. SEQ.ID.-4, SEQ.ID.-5, SEQ.ID.-6, SEQ.ID.-7, SEQ.ID.-8, inhibits α-synuclein fibrillation. The α-synuclein fibrillation was monitored using ThioflavinT (ThT) dye, which specifically binds to amyloids, with its subsequent increase in fluorescence intensity recorded at 482 nm. The purified α-synuclein (400 μM) was incubated at 37° C. with shaking, in the presence and absence of equimolar concentration of indicated peptides. Amyloid formation was determined by monitoring ThT fluorescence at different time intervals. The reduction in ThT fluorescence, as observed upto 6 hr, suggests that the peptides inhibit α-syn fibril formation.

FIG. 4A. The inhibition of α-synuclein fibrillation in the presence of different peptides as indicated by SEQ.IDs. The data is same as in FIG. 3A except that only the enhanced (zoomed) Y-axis for α-synuclein fibrillation in the presence of peptides is shown for clarity. As shown, as compared to penetratin, the SEQ.ID.-7 and SEQ.ID.8 did not allow increase in α-synuclein fibrillation as evident by insignificant increase in fluorescence intensity.

FIG. 4B. The amplitude of fluorescence intensity at 0 h and 5 h were taken from the data shown in FIG. 3A. The ratio of the amplitude at 5 h versus 0 h for each of the curve was calculated and normalized. As shown the peptide derivatives of Seq.ID.-7 and Seq.ID.-8 are significantly better inhibitor of fibrillation than of SEQ.ID.-4, as no much change in fluorescence intensity relative to that at the beginning of fibrillation reaction was observed when α-synuclein fibrillation was carried out in the presence of these derivatives.

FIG. 5A. The sequence alignment showed ˜83% homology (identity) between SEQ. ID.-4 and SEQ. ID.-7.

FIG. 5B. The sequence alignment showed ˜75% homology (identity) between SEQ. ID.-4 and SEQ. ID.-8.

### DETAILED DESCRIPTION OF THE INVENTION

Many of the amyloid based disorders are neurodegenerative in nature. Thus one of the primary challenge in the design of therapeutics against amyloid diseases has been to develop inhibitor that could not only prevent the formation of amyloid fibrils but also able to cross the blood brain barrier. Thus the main objective of present study is the identification of peptide permeable to blood brain barrier that could inhibit the fibrils formation by intrinsically disordered protein α-synuclein. Based upon previous reports that few CPPs are capable of crossing BBB, we explored their potential for the inhibition of fibril formation.

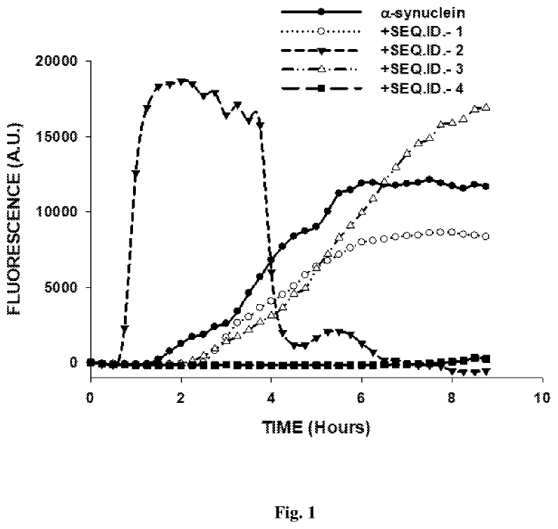

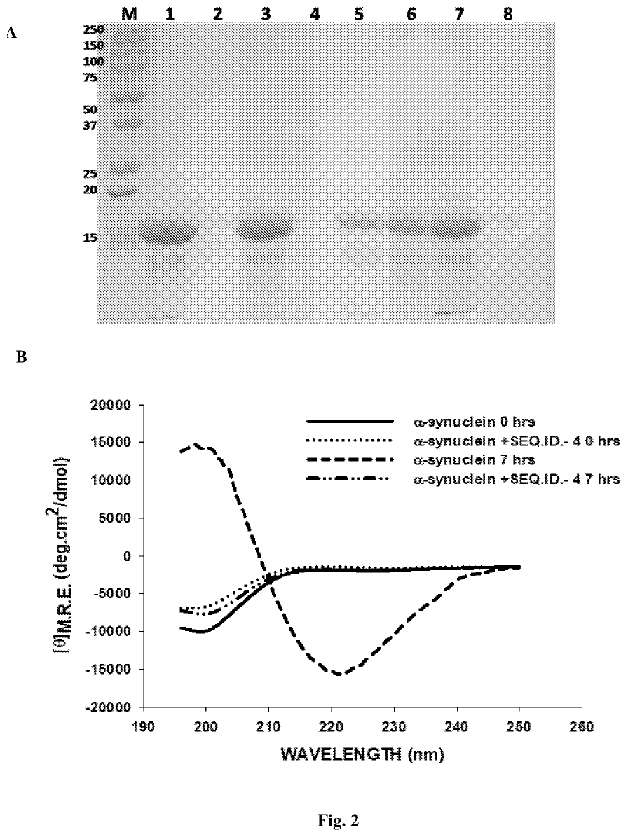

The intrinsically disordered protein, α-synuclein was purified and an assay was established to monitor its fibrillation. We show that α-synuclein, when incubated at 37° C. with shaking conditions starts forming fibrils. We next examined α-synuclein fibril formation in the presence and absence of various CPPs such SEQ.ID.-1, SEQ.ID.-2, SEQ.ID.-3 and SEQ.ID.-4. As shown in FIG. 1, SEQ.ID.-4 inhibited fibrillation of α-synuclein significantly (approximately 90%), while the fibrillation was not much affected in the presence of other CPPs. In order to further confirm that the decrease in Thioflavin T fluorescence in the presence of SEQ.ID.-4 was due to its ability to inhibit α-synuclein fibrillation, the solubility of fibril forming protein was examined on SDS-PAGE. The sample containing α-synuclein incubated at 37° C. in the presence or absence of equimolar concentration of SEQ.ID.-4 was collected at the initiation (0 hrs) or after 7 h of incubation. The samples were fractionated into supernatant and pellet, and the fractions were separated using 15% SDS-PAGE. As shown in FIG. 2a, though α-synuclein was found to be primarily in the supernatant in the beginning of the incubation, more than 50% of the protein precipitated in the pellet fraction after 7 hrs. Interestingly the reaction containing co-incubation with SEQ.ID.-4 contains almost all of α-synuclein in the supernatant at both time points suggesting that the addition of SEQ.ID.-4 prevents aggregation of α-synuclein into amyloid fibrils and helps the intrinsically disordered protein to remain in the soluble fraction.

It is known that the intrinsically disordered protein α-synuclein which lacks characteristics of regular secondary structure gets converted into β-sheet rich amyloid fibrils. Thus the change in β-sheet content is used as a measure to monitor the presence of the amyloid fibrils. In order to further investigate that SEQ.ID.-4 inhibits formation of α-synuclein fibrils in vitro, secondary structure of α-syunclein with and without SEQ.-ID.-4 were analysed using CD. The α-synuclein was incubated at 37° C. in the presence and absence of SEQ.ID.-4, and CD spectra were recorded from aliquots collected before and after fibrillation. As seen in FIG. 2B, aliquots collected after fibrillation show characteristics of β-sheet or disordered secondary structure depending upon whether SEQ.ID.-4 is absent or present in the reaction, clearly suggesting that the peptide inhibits α-synuclein fibrillation and thereby promotes its native disordered structural content.

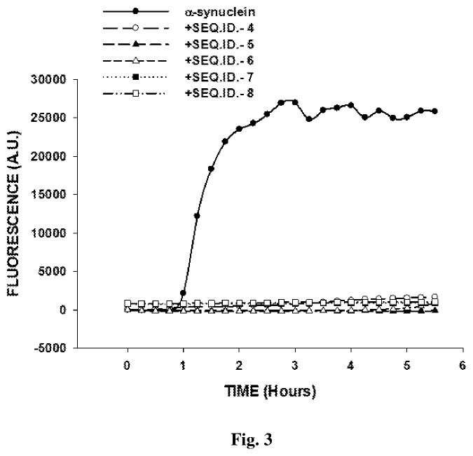

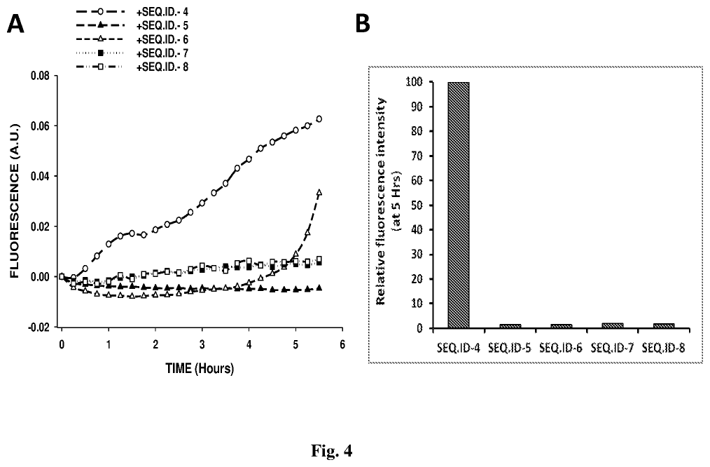

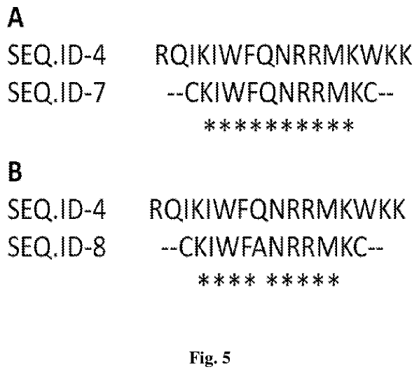

Generally the major drawback in use of peptides as drug is their poor pharmacokinetics. In order to increase stability of SEQ.ID.-4, N-terminal and C-terminal capping was done by acetylation and amidation respectively (SEQ.ID.-5). Also Cyclic derivative (SEQ.ID.-6) synthesized by introducing Cys residue at N-terminal and C-terminal of SEQ.ID.-4 sequence. The SEQ.ID.-4 like peptides were checked for defibrillation activity by incubating with α-syn and carrying ThioflavinT fibrillation assay. To increase the cost effectiveness and activity, several shorter truncated versions of SEQ.ID.-4 were identified and further cyclized to obtain SEQ.ID.-7 and SEQ.ID.-8. SEQ.ID.-8 also has an additional mutation in addition to being a shorter and cyclized derivative. The activities of all derivatives to inhibit α-synuclein fibrillation were confirmed using ThT based fluorescence assay. FIG. 3 shows that SEQ.ID.-4 like peptides are also capable of inhibiting α-synuclein fibrils. Collectively these results suggest that SEQ.ID.-4 and its derivatives has high potential as a lead therapeutic molecule for the treatment of various disorders caused by accumulation of α-synuclein amyloid fibrils. Further, the ability of all derivatives to inhibit α-synuclein fibrillation was compared by measuring the fluorescence change before and after α-synuclein fibrillation (FIG. 4). The fluorescence intensity at 5 h was normalized with respect to that in the beginning of the reaction. FIG. 4B compares the normalized fluorescence intensity of fibrillation reaction in the presence of SEQ.ID.-4 with that obtained in the presence of its other derivatives. The analysis showed that peptides corresponding to SEQ.ID.-7 and SEQ.ID.-8 are better inhibitors than SEQ.ID.-4 against α-synuclein fibrillation.

SEQ.ID.-4 is a well-known CPP (Turner, J. J. et al. Cell-penetrating peptide conjugates of peptide nucleic acids (PNA) as inhibitors of HIV-1 Tat-dependent trans-activation in cells. Nucleic Acids Research 33, 6837-6849, 2005 (doi: 10.1093/nar/gki991) and has been shown to cross BBB (Bolton, S. J. et al. Cellular uptake and spread of the cell-permeable peptide penetratin in adult rat brain, European Journal of Neuroscience 12, 2847-2855, 2000 (doi: 10.1046/j.1460-9568.2000.00171.x) and Bera, S., Kar, R. K., Mondal, S., Pahan, K. & Bhunia, A. Structural Elucidation of the Cell-Penetrating Penetratin Peptide in Model Membranes at the Atomic Level: Probing Hydrophobic Interactions in the Blood-Brain Barrier. Biochemistry 55, 4982-4996, 2016 (doi: 10.1021/acs.biochem.6b00518), which suggests that it can be used as a cargo to deliver neuroprotective agents to brain (Meade, A., Meloni, B., Mastaglia, F. & Knuckey, N. The application of cell penetrating peptides for the delivery of neuroprotective peptides/proteins in experimental cerebral ischaemia studies. JOURNAL OF EXPERIMENTAL STROKE AND TRANSLATIONAL MEDICINE 2, 22-40, 2009 and Joliot, A. & Prochiantz. A. Transduction peptides: from technology to physiology. Nat Cell Biol 6, 189-196, 2004. There are prior arts which states carrier ability of SEQ.ID.-4 to deliver anti-cancer (Fåhracus, R., Paramio, J. M., Ball, K. L., Laín, S. & Lane, D. P. Inhibition of pRb phosphorylation and cell-cycle progression by a 20-residue peptide from p16CDKN2/INK4A. Current Biology 6. 84-91, 1996 doi: https://doi.org/10.1016/S0960-9822(02)00425-6) and anti-amyloid drugs. In regard with usc against α-synuclein, there are prior arts which show that α-synuclein has ability to interact with TAT-derived CPP (Zigoneanu, I. G. & Pielak, G. J. Interaction of α-Synuclein and a Cell Penetrating Fusion Peptide with Higher Eukaryotic Cell Membranes Assessed by 19F NMR. Molecular Pharmaceutics 9, 1024-1029, 2012, doi: 10.1021/mp200615m). Few prior arts also report peptide sequences which act against α-synucleinfibrillation (WO2004009625 and Shaltiel-Karyo, R. et al. Inhibiting α-Synuclein Oligomerization by Stable Cell-Penetrating β-Synuclein Fragments Recovers Phenotype of Parkinson's Disease Model Flies. PLOS ONE 5, e13863, 2010, doi: 10.1371/journal.pone.0013863). Based on these prior arts we can state that CPPs have been widely used as cargoes to deliver drugs to various mammalian tissues including brain implying that it is not toxic to cells. Also researchers have reported peptide sequences acting against α-synuclein amyloidogenesis, but they lack ability to cross BBB. Thus, ability of a peptides of Formula 1 (X₁[RQI]ₘKIWFX₂NRRMK[WKK]ₘX₃) to act as a potent inhibitor of amyloid formation of α-synuclein is very useful in drug development against Parkinson's Disease.

### EXAMPLES

The following examples are given by way of illustration therefore should not be construed to limit the scope of the Invention. The peptides can be used in pharmaceutical compositions such as lipidic formulations like liposomes, solid lipid nanoparticles with peptide stability and penetration enhancers.

### Example 1: Expression and Purification of α-synuclein

The human wild-type α-synuclein protein was expressed in E. coli Rossetta(DE3) using pT7-7 based expression vector. After IPTG induction bacterial cell pellets were harvested by centrifugation and resuspended in 10 mMTris-HCl, pH 8.0, 1 mM EDTA, 1 mM Pierce protease inhibitor cocktail. Protein purification was performed with minor changes in protocol as mentioned previously (van Raaij, M. E., Segers-Nolten, I. M. J. &Subramaniam, V. Quantitative Morphological Analysis Reveals Ultrastructural Diversity of Amyloid Fibrils from α-Synuclein Mutants. Biophysical Journal 91, L96-L98, 2006, doi: 10.1529/biophysj. 106.090449). Briefly, cell lysis was carried out by sonication, lysate was boiled at 95° C. for 30 minutes, followed by centrifugation at 10,000×g for 30 minutes at 4° C. Streptomycin sulfate and Glacial acetic acid precipitated DNA was removed by centrifugation at 13,500×g for 30 minutes at 4° C. 50% ammonium sulfate was added to selectively precipitate the α-synuclein protein by incubation on ice for 1 hour with regular shaking after every 10 minutes. The pellet was separated by centrifugation at 13,500×g for 30 min. at 4° C. and washed with equal volume of 100 mM ammonium acetate followed by equal volume of ethanol. Pellet was dissolved in 10 mM HEPES, 50 mM NaCl, pH 7.4 and dialysed extensively to remove ammonium sulphate. Protein purity was confirmed on 15% SDS-PAGE.

### Example 2: Peptide Synthesis

SEQ.ID.-1, SEQ.ID.-2, SEQ.ID.-3, SEQ.ID.-4, SEQ.ID.-5 and SEQ.ID.-6 were synthesized at peptide synthesizing facility at our Institute of Microbial Technology, Chandigarh, India. Peptides were synthesized by solid phase peptide synthesis strategy using Fmoc (N-(9-fluronyl)-methoxycarbonyl) chemistry in 0.01 mmole scale on a Protein Technologies Inc, USA, PS-3 peptide synthesizer as described (Gautam, A. et al. Identification and characterization of novel protein-derived arginine-rich cell-penetrating peptides. European Journal of Pharmaceutics and Biopharmaceutics 89, 93-106, 2015, doi: http://dx.doi.org/10.1016/j.cjpb.2014.11.020). Other truncated derivatives of SEQ.ID.-4 (SEQ.ID.-7 and SEQ.ID.-8) were synthesized and obtained from Genscript (Piscataway, NJ, USA).

### Example 3: In Vitro Fibrillation Assay

Thioflavin T (ThT) (4 mM) was added to 400 μM of purified α-synuclein with and without Cell penetrating peptides at equimolar concentration in a 96 microwell plate. The plate was incubated at 37° C. with a shaking speed of 900 rpm in linear mode in a multimode plate reader (TECAN infinite M200 PRO). Fluorescence kinetics was measured after every 15 minutes with emission wavelength of 482 nm upon excitation at 442 nm. Each experiment was repeated at least three times.

### Example 4: SDS-PAGE Analysis

The SDS-PAGE analysis was carried out using 15% Tris-bisacrylamide gels operated at a constant current of 30 mA. The running buffer was 25 mMTris-HCl, 193 mMglycine, and 0.1% SDS (pH 8.3). Samples were taken before (0 hrs) and after fibrillation (7 hrs), and fractionated by centrifugation at 4000 rpm for 1 minute into supernatant and pellet. Each fraction was resuspended into 1×SDS loading dye and further separated on 15% SDS-PAGE. The protein bands were visualized by staining SDS-PAGE with 0.1% Coomassie Blue G-250.

### Example 5: CD Analysis

Far UV CD spectra were recorded on a JASCO-J-815 spectropolarimeter. The spectra were recorded over 250-195 nm with a 1-mm pathlength cuvette with scan rate of 10 nm/min with step size of 1 nm at 25° C. and averaged over three scans. The raw CD data were converted into Mean Residue ellipticity (θ_{MRE}), expressed as degrees square centimeter per decimole as follows:

θ_{MRE}=(100*θ_{obs})/[d*C*(n−1)]

Where θ is the observed ellipticity (in degrees), d is path length (in centimeters), C is protein concentration (molar), and n is the total number of amino acids in the protein.

### Advantages of the Invention

1. We have reported that the cell-penetrating peptide SEQ.ID.-4 and its derivatives have the ability to inhibit α-synuclein fibrillation while the other CPPs tested in this study are unable to do so.
2. Since it has been reported that SEQ.ID.-4 can cross the BBB, it and its derivatives have high potential to be a potent lead therapeutic molecule for the treatment of amyloid based diseases.
3. Although SEQ.ID.-4 has been used as a vehicle for drug delivery in various mammalian tissues including brain, but efficacy of SEQ.ID.-4 alone against neurodegenerative disorders had never been studied. The present invention reports the novel property of the peptide inhibition of α-synuclein protein aggregation by SEQ.ID.-4.
4. SEQ.ID.-4 is already known to cross BBB and it is well established that SEQ.ID.-4 is not toxic to mammalian cells thus, its direct application as anti-amyloid drug has high therapeutic value.
5. The novels derivatives SEQ.ID.-7 and SEQ.ID.-8 are found to be more potent than SEQ.ID.-4 in inhibition of α-synuclein fibrillation, and thus could be better anti-amyloid drugs.

1. A peptide having an amino acid sequence of Formula 1 X₁[RQI]ₘKIWFX₂NRRMK[WKK]ₘX₃ and its derivatives thereof wherein
each of X₁ and X₃ is Cysteine or Cystine,
X₂ is Gln or Ala, and
m=0 or 1,
or a peptide having SEQ. ID. NO. 7 or SEQ. ID. NO. 8, each of which is a truncated version of SEQ. ID. NO. 4 having the homology of 83% and 75% respectively.

2. (canceled)

3. The peptide as claimed in claim 1, wherein the N-terminus is acetylated and C-terminus is amidated.

4. A medicament for treating amyloid disorder comprising a peptide of claim 1.

5. A method of determining inhibition of α-synuclein aggregation amyloid by a peptide having amino acid sequence of Formula 1 according to claim 1, the method comprising:
(a) mixing the peptide with α-synuclein to obtain a sample,
(b) mixing the sample as obtained in step (a) with thioflavin T dye to obtain a purified α-synuclein;
(c) incubating the purified α-synuclein as obtained in step (b) at room temperature or 37° C. under shaking conditions, and
(d) measuring the fluorescence intensity at 482 nm to monitor the amyloid formation.

6. A pharmaceutical composition comprising a peptide of formula 1 in according to claim 1 and a pharmaceutically acceptable excipient.

7. A method of treating an α-synuclein amyloid associated disorder in a patient comprising administering to the patient an effective amount of a peptide of claim 1, or a pharmaceutical composition thereof.

8. The method of claim 7, wherein the disorder is Parkinson's disease or another form of α-synucleinopathy.
